(19) United States
(12) Patent Application Publication
(10) Pub. No.: US 2025/0381325 A1
(43) Pub. Date: Dec. 18, 2025

(54) SINGLE UNIT BLOOD WARMER

(71) Applicant: Delta Development Team, Inc., Tucson, AZ (US)

(72) Inventors: William Barg, Tucson, AZ (US); Christopher Bridgewater, Tucson, AZ (US); Robert Futch, Tucson, AZ (US); Montgomery Leija, Sahuarita, AZ (US)

(21) Appl. No.: 19/082,628

(22) PCT Filed: Sep. 18, 2023
(86) PCT No.: PCT/US23/74461

§ 371 Date: Mar. 18, 2025

Related U.S. Application Data
(60) Provisional application No. 63/408,779, filed on Sep. 21, 2022.

(51) Int. Cl. A61M 1/02; A61M 1/36

(52) U.S. Cl. CPC A61M 1/0281; A61M 1/369; A61M 2202/0415; A61M 2205/075; A61M 2205/3331; A61M 2205/3368; A61M 2205/3673; A61M 2205/8206

(57) ABSTRACT

Systems, methods, and devices to warm and deliver blood and plasma to patients may include a body, an infusion sleeve coupled to the body and encircling a volume, and a control panel disposed on the body. A power source is in electronic communication with the control panel, and the power supply may be a battery external to the body. A heater is in electronic communication with the battery and disposed adjacent an interior surface of the infusion sleeve. The heater comprises a plurality of heating elements and a plurality of temperature sensors configured to measure a temperature of each heating element from the plurality of heating elements. Power delivered to each heating element may be adjustable in response to the temperature of the heating element or an estimated temperature of a blood or plasma bag.

### CROSS-REFERENCE TO RELATED APPLICATION

This application claims priority to Patent Cooperation Treaty (PCT) Application No. PCT/US23/74461 filed on Sep. 18, 2023 and entitled, “SINGLE UNIT BLOOD WARMER,” which claims the benefit of U.S. Provisional Patent Application No. 63/408,779 filed on Sep. 21, 2022 and entitled, “SINGLE UNIT BLOOD WARMER,” both of which are incorporated herein by reference.

### GOVERNMENT LICENSE RIGHTS

This invention was made with government support under FA8629-20-C-5023 awarded by United States Air Force, Air Force Lifecycle Management Center (“AFLCMC”). The government has certain rights in this invention.

### FIELD

The present disclosure relates to thermal management for transportation and administration of blood transfusions, and more particularly to warming blood.

### BACKGROUND

Medical conditions may not always arise in ideal conditions, and a hospital may not be available when they do. A patient in the field may suffer conditions that merit emergent treatment with advanced techniques typically only available in a hospital or treatment facility. A wounded individual may be treatable with a blood transfusion, for example, in a hospital or other facility with the ability to maintain donor blood.

However, some techniques of modern medicine may be unavailable in the field due to temperature, climate, or other environmental factors. Blood is temperature sensitive. Refrigeration systems are commonly used to preserve blood during transportation prior to transfusion. However, chilled blood is too cold for transfusion.

Blood is typically warmed to a suitable temperature prior to administering blood to a patient. Available warmers are often single use, necessitating complete or partial replacement after warming a single dose of donor blood. Other warmers may be bulky and suited to use in hospitals or other fixed settings. Still others tend to warm blood unevenly, resulting in inconsistent temperatures across the warmed blood.

### BRIEF DESCRIPTION

Various embodiments relate to blood warming and delivery devices that include Blood and plasma warming device of the present disclosure may include a body, an infusion sleeve coupled to the body and encircling a volume, and a control panel disposed on the body. A power source in electronic communication with the control panel. A heater may be in electronic communication with the battery and disposed adjacent an interior surface of the infusion sleeve. The heater comprises a plurality of heating elements and a plurality of temperature sensors configured to measure a temperature of each heating element from the plurality of heating elements.

Various embodiments include electronic controls configured to control a voltage, current, or power delivered to a heating element from the plurality of heating elements in response to a measured temperature of the heating element. Electronic controls may be configured to control a voltage delivered to a heating element from the plurality of heating elements in response to an estimated temperature of a bag in the volume. A manifold may be in fluid communication with the infuser sleeve and comprising an inlet to receive a hose. The manifold may deliver air from the hose into the infusion sleeve to inflate the infusion sleeve. The battery may be external to the body. A pressure sensor may be in electronic communication with the control panel and configured to measure an air pressure in the infusion sleeve.

Various embodiments of warming device include an infusion sleeve encircling a volume with the infusion sleeve being inflatable to reduce the volume. A heater may be disposed adjacent an inner surface of the infusion sleeve and may comprise a plurality of heating elements. A plurality of temperature sensors may be configured to measure a temperature of each heating element from the plurality of heating elements.

In various embodiments, a heating element from the plurality of heating elements is disposed adjacent the volume and comprises a thermistor trace disposed adjacent the inner surface of the infusion sleeve. Electronic controls may be configured to control a voltage delivered to the heating element in response to a temperature of the heater measured by the thermistor trace. Electronic controls may also be configured to control a voltage delivered to the heating element in response to an estimated temperature of a bag disposed in the volume. The estimated temperature may be based on a temperature of the heater measured by the thermistor trace. A pressure sensor may be configured to measure an air pressure in the infusion sleeve. An infuser bulb may be in fluid communication with the infusion sleeve.

Various embodiments include methods of using a warming device to warm blood or plasma. Methods may include the step of inserting a bag of cooled product into a volume defined by an infusion sleeve. A heater may be disposed about an inner surface of the infusion sleeve. The infusion sleeve is inflated to a first pressure to press the heater against the bag. Heat is applied to the bag through the heater to warm the cooled product. The method further includes inflating the infusion sleeve to a second pressure to urge the warmed product from the bag.

Embodiments include steps of coupling an infusion hose to the bag after applying heat to the bag and before inflating the infusion sleeve to urge the warmed product from the bag. The first pressure may be in a range from 150 mmHg to 300 mmHg. The heater may be powered by a battery in electronic communication with the heater. The method may automatically adjust a voltage delivered to the heater in response to a temperature measured at the heater. The method may also adjust a voltage delivered to the heater in response to an estimated temperature of the cooled product.

### DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures, wherein like numerals denote like elements.

FIGS. 1A and 1B illustrate perspective view of a blood warming and delivery device, in accordance with various embodiments.

FIG. 2 illustrates a control panel of a blood warming and delivery device, in accordance with various embodiments.

FIG. 3 illustrates a perspective cutaway view of a blood warming and delivery device, in accordance with various embodiments.

FIG. 4 illustrates a side cutaway view of a blood warming and delivery device defining a volume for heating, in accordance with various embodiments.

FIG. 5 illustrates a schematic view of a heater for a blood warming and delivery device, in accordance with various embodiments.

FIG. 6 illustrates an arrangement of heating elements and sensors for a blood warming and delivery device, in accordance with various embodiments.

### DETAILED DESCRIPTION

The following detailed description is intended to provide several examples that will illustrate the broader concepts set forth herein, but it is not intended to limit the invention or applications of the invention. Furthermore, there is no intention to be bound by any theory presented in the preceding background or the following detailed description.

The present disclosure relates to battery powered, portable, blood and plasma warming devices. These devices are suitable for military or civilian use. Warming devices of the present disclosure may operate in the absence of water for thawing. An infusion kit may be used in warming devices for sterile delivery. Single Unit Blood Warmers (SUBW) of the present disclosure may continue to warm bags of plasma or blood repeatedly with reusable electronics, which differs from devices that operate using disposable electronics.

Blood warmer devices of the present disclosure are blood warming and plasma thawing devices that include integrated pressure infusers in various embodiments. Blood warmers of the present disclosure warm blood products prior to infusion to prevent hypothermia in recipients. SUBW devices are suitable for use by healthcare professionals in hospital, clinical, field and transport environments. The SUBW is capable of operation in severe environments with exposure to rain, dust, rough handling, and extremes in temperature and humidity.

SUBW devices may use multiple heaters to deliver fast, even heating application. An array of sensors may be disposed about SUBW devices to enable independent assessment and control of heaters. Sensors may indirectly measure temperature of heaters or blood bags.

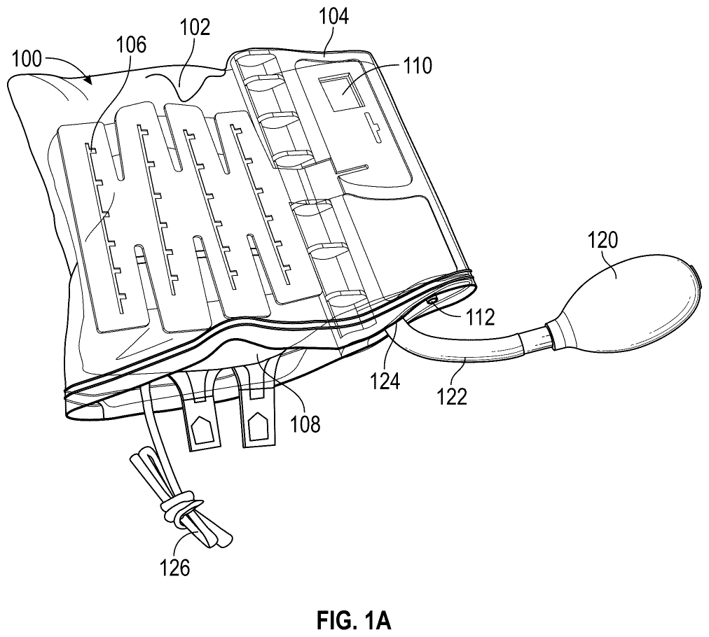

With reference to FIGS. 1A and 1B, SUBW 100 is shown, in accordance with various embodiments. FIG. 1A includes depicts device 100 including protective layer 103, and FIG. 1B depicts device 100 with protective layer 103 removed. SUBW 100 includes infuser sleeve 102 coupled to body 104. Body may serve as a housing to retain and protect electronics and pneumatics and to serve as a rigid support member to maintain a shape of the flexible infuser sleeve 102. Infuser sleeve 102 retains blood bag 108. Blood bag 108 may be inserted into infuser sleeve 102 by sliding in from the top or bottom of infuser sleeve 102. Heater 106 is disposed about the interior of infuser sleeve 102. Heater 106 may be proximate to or contacting blood bag 108 to transfer heat from heater 106 to blood bag 108. Heater 106 may be flexible and may include heat-conductive materials such as, for example, copper, polyimide, flexible circuits, multi-layer flexible circuits, or other flexible materials and material combinations capable of transferring heat. Heater 106 typically generates resistive heat by dissipating electrical energy. Infuser sleeve 102 may be inflatable to apply pressure to blood bag 108 or to urge heater 106 closer to blood bag 108. For example, infuser sleeve 102 may be pressurized to 150 mmHg-300 mmHg to facilitate heating.

Infuser sleeve 102 may be made from a flexible material. Infuser sleeve may be made of plastic or rubber, for example. Infuser sleeve 102 may be surrounded by or may include protective layer 103 comprising a protective material disposed about that outer surface of the infuser sleeve to protect infuser sleeve 102 from punctures, cuts, abrasion, or other damage that might inhibit operation. The protective material of protective layer 103 may include an abrasion or cut resistant textile, canvas, synthetic fibers, Kevlar®, aramid fibers, rubber, plastic, woven metal, or other material suitable for protecting infuser sleeve 102. Infuser sleeve 102 defines an airtight cavity to facilitate inflation, with pneumatic passages into body 104. Body 104 may retain an edge of infuser sleeve 102. A valved manifold or valved tubing may be retained in body 104 to facilitate inflation and deflation of infuser sleeve 102.

In various embodiments, body 104 may be rigid or semi-rigid to retain electronics and pneumatic components. Body 104 may thus be made of plastic, metal, rubber, or other materials suitable for retaining electronics and pneumatic components. Body 104 includes control panel 110, which may further include input and output mechanisms for outputting data and controlling operation of SUBW 100. Body 104 houses a battery in some embodiments and power input 112 to receive a charging device to charge the battery. In some embodiments, the battery is external to body 104. SUBW 100 may be compatible with 120V or 240V charging devices that adapt electrical output in alternating current to a suitable direct current for charging. For example, adapters may deliver 24 volts of direct current to SUBW 100 for charging or operation from an alternating current power supply. In another example, SUBW 100 may accept between 12 to 30 VDC at power input 112.

In various embodiments, SUBW may be compatible with an infusion kit to facilitate transfusion from blood bag 108 withing SUBW 100. Hose 122 may be coupled to body 104 by mating interface 124. Infuser bulb 120 is coupled to hose 122 to deliver pressurized air through hose 122, into pneumatic components contained in body 104, and into infuser sleeve 102 to inflate infuser sleeve 102. Infuser sleeve may apply compressive force to blood bag 108 in response to inflation. Compressive force applied by infuser sleeve 102 may urge warmed blood from blood bag 108 through hose 126 to patient.

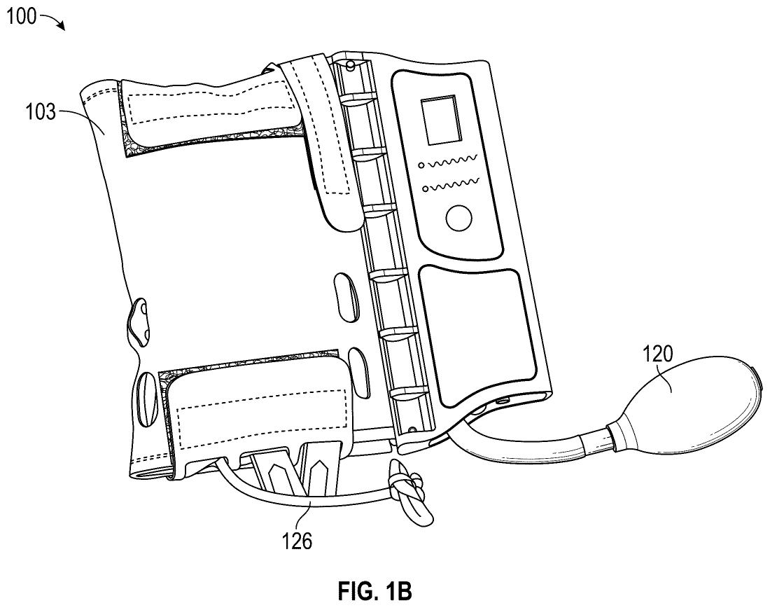

Referring now to FIG. 2, control panel 110 is shown, in accordance with various embodiments. Control panel 110 includes display 200 to print information related to operations. Display 200 may be an LED, LCD, OLED, or other type of screen suitable for outputting information regarding operation of SUBW 100. Display 200 may be configured to show a temperature of heater 106, a temperature of blood bag 108 (of FIG. 1), a pressure of infuser sleeve 102, time remaining in heating operation, time remaining in transfusion operation, or other information related to warming and delivering blood or plasma.

In various embodiments, control panel 110 may comprise one or more button 202. Button 202 enables control of SUBW 100. Button 202 may cycle through operating settings or display settings of SUBW 100. Button 202 may open and close power delivery circuits that deliver electricity to heater 106, which may use resistive heating elements to convert electricity into heat. Bridge 212 may contain traces or electrodes that selectively deliver power to heating elements in heater 106.

In various embodiments, control panel 110 may also include indicator lights. Indicator lights may indicate battery charge level, errors, power status, or other information. SUBW may include a speaker to generate audible alarms in response to detecting abnormal operating conditions. Alarms and alerts may be silenced for 120 seconds on startup to allow SUBW 100 to reach nominal operating condition without throwing false alarms or alerts. Abnormal operating conditions may include low battery power, unexpected high or low temperatures detected on heater 106, charging fault, or other abnormal operating conditions typical of battery-operated devices.

In various embodiments, body 104 may include mating interface 204 to retain infuser sleeve 102. Mating interface 204 as depicted includes ridge 208 extending parallel to an edge of body 104. Protrusions 206 may be oriented perpendicular to ridge 208 to increase rigidity and strength of mating interface 204. Mating interface 124 securely receives and securely retains mating edge 210 of infuser sleeve 102. Mating interface 124 may be a clamp, channel, groove, or other mating interface suitable for coupling and retaining infuser sleeve 102 in place relative to body 104. Electronics and pneumatic components may thus pass from body 104 to infuser sleeve 102 at substantially fixed positions relative to infuser sleeve 102. Body 104 defines opening 114 for hanging SUBW 100. A coat hanger, strap, hook, post, or other hanging device may pass through opening 114 to support SUBW 100 in a hanging position. In embodiments with protective layer 103, opening 114 may be defined by protective layer 103.

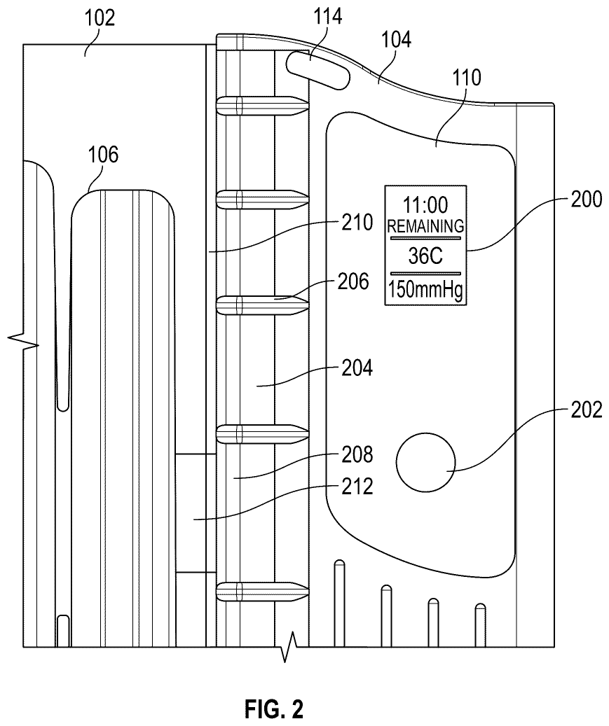

With reference to FIG. 3, SUBW 100 is shown with body 104 cutaway to expose electronics and pneumatic components. Body 104 retains manifold 300 with valving 302 extending into infuser sleeve 102. Manifold may deliver or release air from infuser sleeve 102 to inflate or deflate the infuser sleeve. Hose 122 is in fluid communication with manifold to deliver pressurized air (e.g., from a hand pump) into infuser sleeve 102. Electronics 304 coupled to manifold 300 may include valve controls, valves, pressure sensors, and other mechanical or electrical components that interface with the air delivery and release system.

In various embodiments, body 104 may retain a printed circuit board 306 that interacts with electronic components such as, for example, a battery, sensors, processor, memory, or control panel 110. Substrates 307 may be disposed within body 104 substantially parallel to printed circuit board 306. In some embodiments, substrates 307 may be printed circuit boards, memory, or other electronic devices. Control panel 110 may include indicator lights 308 and 310, which may indicate battery charge level, errors, power status, or other information.

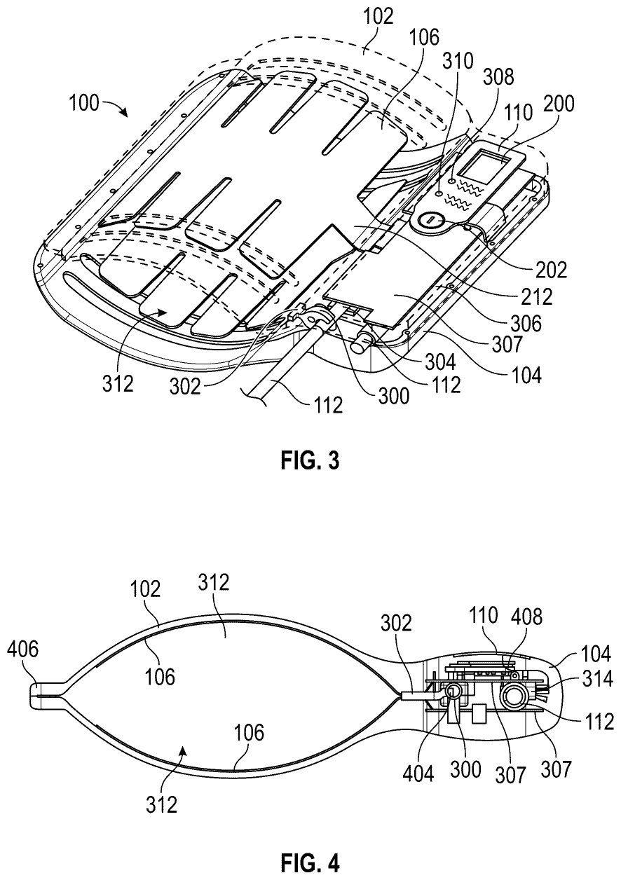

With reference to FIG. 4 and continuing reference to FIG. 3, bridge 212 may carry power from a battery in body 104 through conduits such as traces in printed circuit board 306 or substrate 307, wires, or electrical terminals to heater 106. Bridge 212 may thus include a pair of electrodes or traces for each independently controllable heating element of heater 106. Heaters 106 may be disposed about the inner diameter of infuser sleeve 102. Stated another way heaters 106 may be disposed on opposing internal surfaces of infuser sleeve 102. Infuser sleeve 102 may include end 406 that is fused, coupled, fixed, or otherwise bound to encircle volume 312 with an open top and bottom to receive blood bag 108 (of FIG. 1).

In various embodiments, manifold 300 includes air inlet 404 suitable for mating with hose 122. Air inlet 404 receives air for delivery into infuser sleeve 102 through valving 302. Valving 302 may include a check valve in fluid communication with air inlet 404 to restrict the air from escaping through air inlet 404, though in many embodiments inflation bulb 120 (of FIG. 1) contains the check valve in fluid communication with air inlet 404. An overpressure valve may also be in fluid communication with manifold 300 to selectively release air from infuser sleeve 102. In some embodiments, the overpressure valve is coupled to or formed integrally with the manifold (e.g., in valving 302 of FIG. 3). The overpressure valve bleeds air in response to high pressures in infuser bag 102. The overpressure valve may be mechanically or electronically actuated by manual user selection, by electronic controls 408, or by mechanical configuration.

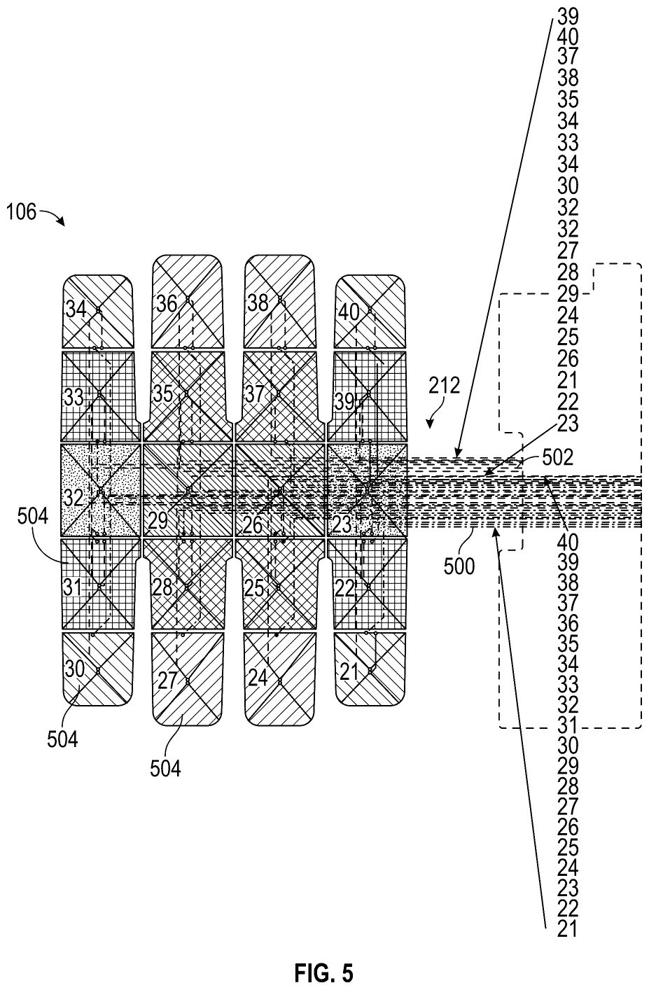

Referring now to FIG. 5, heater 106 is shown, in accordance with various embodiments. Heater 106 may include multiple heating elements 504 (numbered 21 through 40). Each heating element may have a negative lead 500 and a positive lead 502. The leads may be traces embedded in or disposed on heater 106. Heater 106 is depicted with 20 independently controllable heating elements =, though any number of independently controllable heating elements may be used.

In various embodiments, SUBW 100 may include two heaters 106. For example, a SUBW using two heaters 106 may have forty independently controllable heating elements. Each heating element may include a thermistor to measure the temperature at the heating zone. The measured heat from a thermistor may be used to reduce or increase power delivery over the positive lead 502 and negative lead 500 corresponding to the thermistor.

SUBW 100 using 40 heating elements and 40 sensors, for example, may include two heaters 106 arranged as shown in FIG. 5 to selectively deliver power to heating elements. SUBW 100 may deliver more power to the elevated portion of infuser sleeve 102. Accelerometers or position sensors may be used to determine the orientation of SUBW 100 and of infuser sleeve 102. The lowest position of infuser sleeve 102 may also be detected using the thermistors to detect the coolest part of blood bag 108, as the coolest part is lowest relative to gravity during heating. SUBW 100 may use heater 106 to maintain desired heat levels at each heating zone.

Although 20 heating elements 504 per heater 106 is given as an example, each heater 106 may have any number of heating elements 504. SUBW 100 may use the heating elements 504 to keep the entire heating plate at a substantially uniform temperature. Multiple independently controlled heating elements may enable SUBW 100 to maintain a desired temperature across heater 106 more uniformly than heaters that use a single heating element. For example, heating element 504 may be maintained at 45° C., 46° C., 47° C., 48° C., 49° C., 50° C., 51° C., or 52° C.

In various embodiments, electronic controls 408 (of FIG. 4) may test circuitry and traces for proper operation. Each heating element 504 and its corresponding positive trace 502 and negative trace 500 may be tested to ensure current flows through each heating element as expected in response to an applied voltage, current, or power. Voltage or current control circuitry may be tested by determining whether a change in the temperature of a heating element 504 matches the expected change in response to a selected amount of energy delivered to heating element 504. An alarm or error may be thrown in response to detecting malfunctioning electronics.

Heater 106 warms bags 108 of blood or plasma quickly because the heating elements 504 are arranged in a grid on each side of the bag 108. SUBW 100 may deliver a different amount of energy to each heater element 504 (i.e., each section of the grid). The amount of energy delivered is based on the convection detected in the bag, as measured by the change in temperature measured by a thermistor located on each heating element 504 (i.e., at each section of the grid). SUBW 100 sends more heat to the colder parts of the bag 108 and as a result warms bag 108 faster than a single heater.

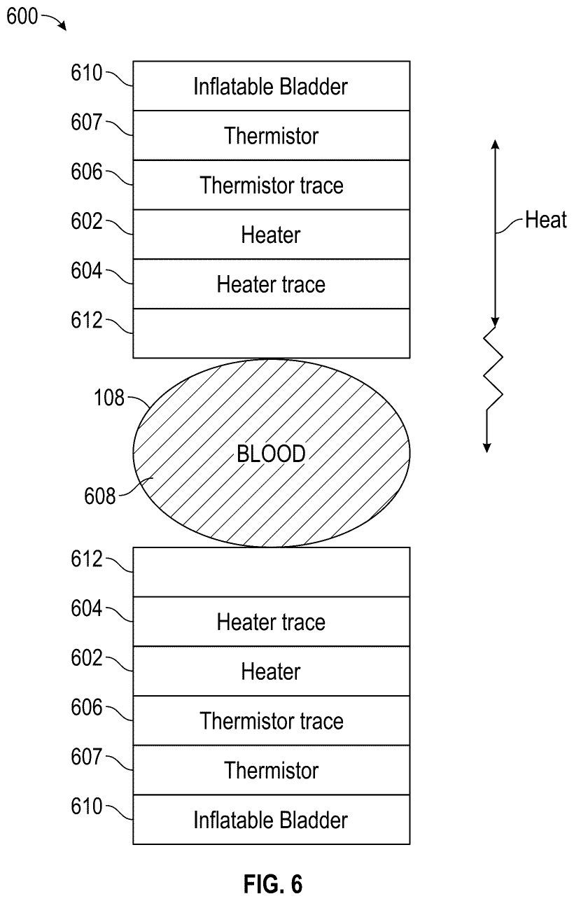

Referring now to FIG. 6, a diagram of heating configuration 600 is shown for use in blood warmers, in accordance with various embodiments. Heating configuration 600 includes a heating element 602 disposed on opposite sides of cooled blood. Heating element 602 includes heater traces 604 disposed on the interior surface of heating element 602. The interior surface of heating element 602 may be adjacent and oriented facing blood bag 108. Heater traces may thus be disposed between blood bag 108 and heating element 602.

In various embodiments, thermal sensors may be disposed on an exterior surface of heating element 602. The exterior surface of heating element 602 may be oriented facing away from blood bag 108. In that regard, the exterior surface of heating element 602 may be adjacent and oriented towards the inner surface of infuser sleeve 102 (not shown). Heating element 602 receives electrical current carried along heater traces and generates heat. The heat generated by heating element 602 permeates inward, towards blood bag 108, and outwards towards the thermistor trace 606 and the infuser sleeve 102. Thermistor trace 606 measures the temperature on the outward-facing surface of heating element 602. Separate thermistor traces 606 and heater traces 604 may be added for each heating element (as shown in FIG. 5).

In various embodiments, inflatable bladder 610 of infuser sleeve 102 (of FIG. 1) is inflatable to press heater 602 towards blood bag 108. A thin layer 612 of plastic, rubber, or other flexible material may be disposed between theater 602 and blood bag 108. Thin layer 612 is typically coupled to or formed integrally with inflatable bladder 610 of infuser sleeve 102. Infuser sleeve 102 may thus define multiple chambers including an inflatable bladder 610 and an area for heater 106 between thin layer 612 and inflatable bladder 610. Thin layer 612 may protect heater 602 from environmental conditions and tends to retain heater 602 in place about volume 312 (of FIG. 3). Thin layer 612 is thin to allow heat transfer across thin layer 612 into bag 108.

Thermistor trace 606 is disposed outside of blood bag 108. Temperature measured at thermistor trace 606 may be used to estimate the temperature of blood or plasma 608 in blood bag 108. Thermistor trace 606 is in electronic communication with thermistor 607 to measure temperature. A temperature measurement of blood bag 108 may be based on the thermal resistance of blood bag 108, the thermal resistance of thin layer 612, the energy in heating element 602, and the temperature measured by thermistor 607. For example, blood temperature, b, may be estimated as b=t−cv²rp where t is the temperature at thermistor trace 606, v is the heater voltage, p is the pulse width modulation (PWM) rate, r is electrical resistance, and c is a constant. Constant c may be determined based on losses to air and thermal resistance of the plastic material of blood bag 108.

SUBW 100 may control power delivered to heating element 602 in response to an estimated temperature of blood 608 neat heating element 602. For example, SUBW 100 (of FIG. 1) may maintain a voltage across heating element 602 to achieve a desired blood temperature b. SUBW may maintain voltage v=sqrt((t−b)/(crp)), where sqrt is the square root function. Voltages corresponding to measured temperatures t may also be retained in a lookup table stored in EPROM, RAM, or other memory suitable for access by processors or other electronic controls 408 (of FIG. 4) in SUBW 100 (of FIG. 1). In that regard, electronic controls 408 (of FIG. 4) may calculate or lookup a voltage for delivery to heating element 602 based on the temperature measured at thermistor trace 606.

In various embodiments, SUBW 100 may operate in harsh prehospital environments and during air and ground transport, as SUBW 100 may continuously monitor blood or plasma temperature and may adjust to hot or cold environments. SUBW 100 may operate in environments from −10° C. to 40° C., from −15° C. to 45° C., or from −20° C. to 50° C., for example. SUBW 100 may also operate from 0 to 5000 meters, from −1000 to 6000 meters, or from −2000 to 7000 meters relative to sea level.

Blood warmers and SUBW devices of the present disclosure may operate without disposable electronics and infuser bags. Devices of the present disclosure may thus continue to warm blood or plasma as long as sufficient power is available. Devices of the present disclosure are also portable relative to some competing products, with SUBW 100 weighing as little as 1 pound. The weight may be further reduced by using lightweight materials and manufacturing techniques.

Blood warmers and SUBW devices of the present disclosure may also function in any orientation, which allows for reliable use in bumpy ambulances. Users need not spend precious time checking on device orientation before warming and delivering blood. Devices of the present disclosure also heat blood or plasma faster than competing devices, with SUBW 100 warming bags in about 10 minutes. SUBW 100 also supports faster infusion flow rates and heating of blood prior to infusion for faster treatment. Blood and plasma may be warmed using SUBW 100 before administration, and SUBW 100 can support quick infusion all of warmed product at rapidly to increase chances of survival. SUBW 100 is also effective warming both blood and plasma for delivery to patients.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the inventions.

The scope of the invention is accordingly to be limited by nothing other than the appended claims, in which reference to an element in the singular is not intended to mean “one and only one” unless explicitly so stated, but rather “one or more.” Moreover, where a phrase similar to “A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

References to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or device that comprises a list of elements does not include only those elements but may include other elements not expressly listed or inherent to such process, method, article, or device.

What is claimed is:

1. A warming device comprising:
a body;
an infusion sleeve coupled to the body and encircling a volume;
a control panel disposed on the body;
a power source in electronic communication with the control panel;
a heater in electronic communication with the battery and disposed adjacent an interior surface of the infusion sleeve, wherein the heater comprises a plurality of heating elements; and
a plurality of temperature sensors configured to measure a temperature of each heating element from the plurality of heating elements.

2. The warming device of claim 1, further comprising electronic controls configured to control a voltage delivered to a heating element from the plurality of heating elements in response to a measured temperature of the heating element.

3. The warming device of claim 1, further comprising electronic controls configured to control a voltage delivered to a heating element from the plurality of heating elements in response to an estimated temperature of a bag in the volume.

4. The warming device of claim 1, further comprising a manifold in fluid communication with the infuser sleeve and comprising an inlet to receive a hose.

5. The warming device of claim 4, wherein the manifold delivers air from the hose into the infusion sleeve to inflate the infusion sleeve.

6. The warming device of claim 4, wherein the power source comprises a battery external to the body.

7. The warming device of claim 4, further comprising an overpressure valve to selectively release air from the infuser sleeve.

8. The warming device of claim 1, further comprising a pressure sensor in electronic communication with the control panel, the pressure sensor configured to measure an air pressure in the infusion sleeve.

9. A warming device comprising:
an infusion sleeve encircling a volume, wherein the infusion sleeve is inflatable to reduce the volume;
a heater disposed adjacent an inner surface of the infusion sleeve, wherein the heater comprises a plurality of heating elements; and
a plurality of temperature sensors configured to measure a temperature of each heating element from the plurality of heating elements.

10. The warming device of claim 9, wherein a heating element from the plurality of heating elements is disposed adjacent the volume and comprises a thermistor trace disposed adjacent the inner surface of the infusion sleeve.

11. The warming device of claim 10, further comprising electronic controls configured to control a voltage delivered to the heating element in response to a temperature of the heater measured by the thermistor trace.

12. The warming device of claim 10, further comprising electronic controls configured to control a voltage delivered to the heating element in response to an estimated temperature of a bag disposed in the volume, wherein the estimated temperature is based on a temperature of the heater measured by the thermistor trace.

13. The warming device of claim 9, further comprising a pressure sensor configured to measure an air pressure in the infusion sleeve.

14. The warming device of claim 9, further comprising an infuser bulb in fluid communication with the infusion sleeve.

15. A method of using a warming device, comprising:
inserting a bag of cooled product into a volume defined by an infusion sleeve, wherein a heater is disposed about an inner surface of the infusion sleeve;
inflating the infusion sleeve to a first pressure to press the heater against the bag;
applying heat to the bag through the heater to warm the cooled product; and
inflating the infusion sleeve to a second pressure to urge the warmed product from the bag.

16. The method of claim 15, further comprising coupling an infusion hose to the bag after applying heat to the bag and before inflating the infusion sleeve to urge the warmed product from the bag.

17. The method of claim 15, wherein the first pressure is in a range from 150 mmHg to 300 mmHg.

18. The method of claim 15, wherein the heater is powered by a battery in electronic communication with the heater.

19. The method of claim 15, further comprising automatically adjusting a voltage delivered to the heater in response to a temperature measured at the heater.

20. The method of claim 15, further comprising automatically adjusting a voltage delivered to the heater in response to an estimated temperature of the cooled product.
